(19) United States
(12) Patent Application Publication
(10) Pub. No.: US 2022/0104826 A1
(43) Pub. Date: Apr. 7, 2022

(54) MULTI-FIRE CLIP APPLIER

(71) Applicant: TransEnterix Surgical, Inc., Morrisville, NC (US)

(72) Inventor: Brian P Cash, Cary, NC (US)

(21) Appl. No.: 17/064,128

(22) Filed: Oct. 6, 2020

(51) Int. Cl. A61B 17/128

(52) U.S. Cl. CPC A61B 17/128; A61B 2017/00367

(57) ABSTRACT

A multi-fire clip applier includes an elongate shaft having a lumen and a jaw assembly on a distal end of the shaft. A plurality of surgical ligating clips are positionable within the lumen. A screw member is rotatable within the lumen to sequentially advance the clips towards the jaw assembly.

### BACKGROUND

Ligature clips, such as the Teleflex Hemolock™ locking polymer clip, are used in various minimally-invasive procedures to ligate tissue in a simple, controlled way. However, currently these clips must be individually loaded into a clip applier apparatus and reloaded after each clip. In some procedures, the use of multiple clips is common, e.g., in a Cholecystectomy, 3 clips are routinely used. While multi-fire mechanisms exist for metal ligating clips, no such device exists for polymer locking clips due in part to their complexity.

### BRIEF DESCRIPTION OF THE DRAWINGS

FIG. 1 is a perspective view of a multi-fire clip applier.

FIGS. 2-4 are cross-sectional side views of the clip applier of FIG. 1, illustrating the sequence of steps for firing a clip.

FIGS. 5 and 6 show the jaws of the clip applier in opened and closed positions, respectively.

FIG. 7 is a distal end view of the clip applier.

FIG. 8 is a perspective view of the rail.

FIG. 9 is a partially disassembled view of the clip applier, in which the tubular member and one of the jaws is not show so as to allow the position of the rail within the device to be seen.

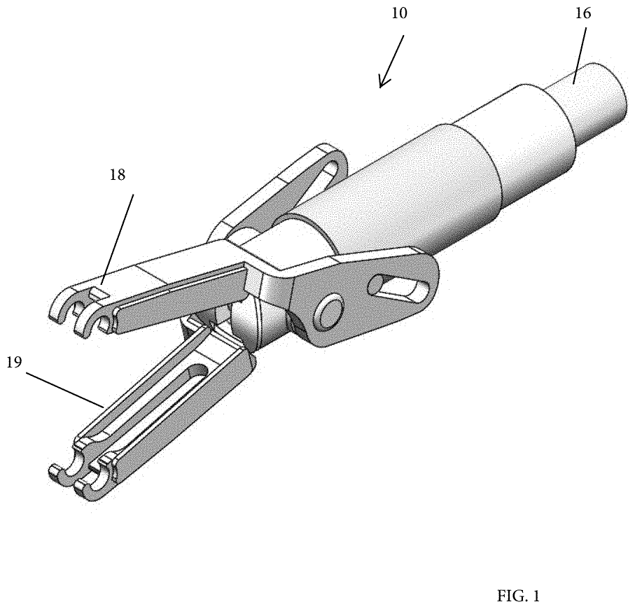

### DETAILED DESCRIPTION

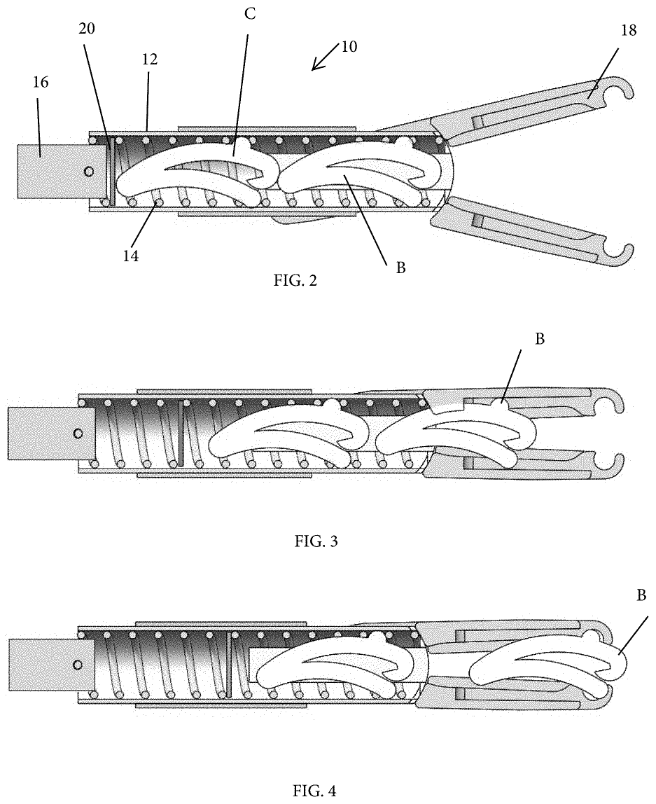

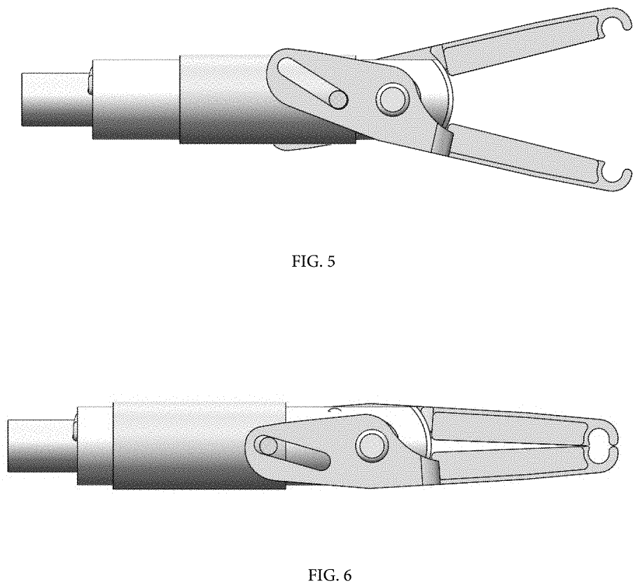

The disclosed multi-fire clip applier is best understood with reference to the sequence of drawings labeled FIGS. 2-4. Clip applier 10 includes an outer tube 12 which houses a spring coil 14 that can be rotated through action of a control mechanism 16. Coil 14 acts as a screw mechanism to sequentially advance the ligating clips C along the tube's axis. In alternate embodiments, alternative helical or screw mechanisms may be used in place of the coil.

Attached to the outer tube is are the clipping jaws 18. Opening and closing of the jaws is effected using a mechanism proximal to the applier, either through linear actuation (e.g., a bowden cable) or through rotary cams driven through a sleeve over the tube. In use, the user actuates the spring coil 14 to advance the distalmost clip C until it engages with or seats against capture features within the clipping jaws. The instrument is then moved into position with the jaws over the tissue to be ligated. The jaws are then closed, locking the clip C using the clipping features of the jaws. Clipping features for clip applier jaws are known to those skilled in the art and typically are specific to the type of clip to be used.

Guides, tracks, or other alignment/capture features in the jaws 18 allow the clip to advance to the clipping features without sliding out of the device. For example, lateral side walls 19 define tracks to prevent the clips from sliding laterally out of the jaws. These tracks could be configured as shown, or could be formed to fully enclose the bosses.

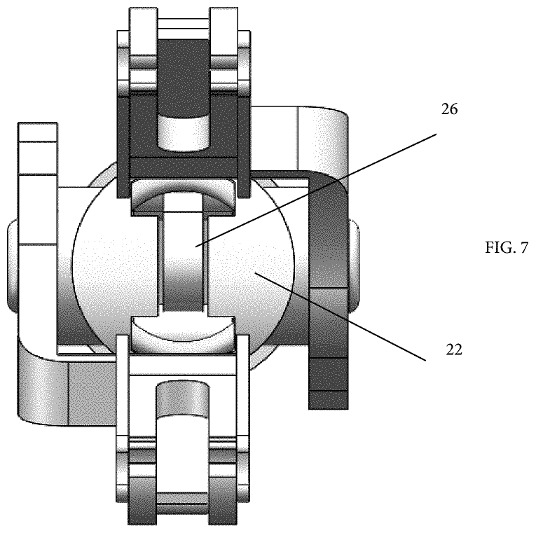

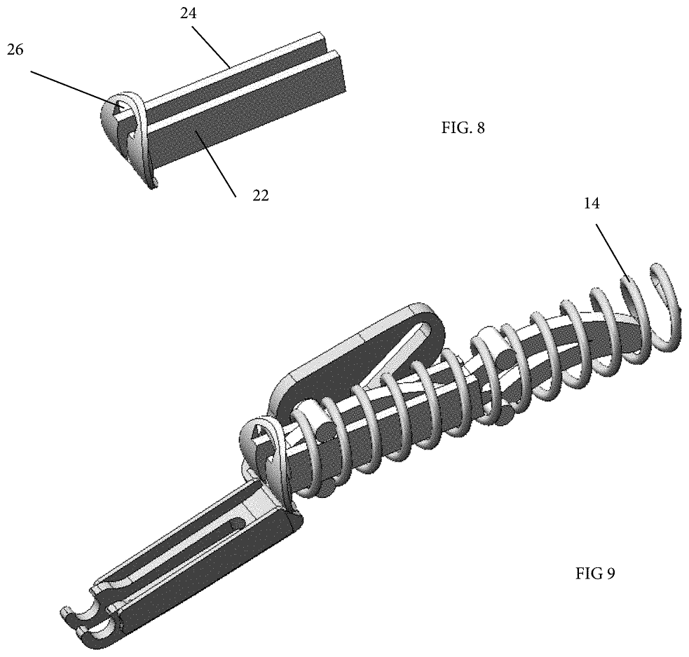

To apply an additional clip, the user actuates the spring to cause the next clip to advance to the jaws. As shown in FIGS. 6-8, with the distal portion of the clip applier shaft is a rail member 22 defining a channel 24 that keeps the distalmost clip in alignment with the jaws. The distal opening of the rail member 22 has a keyhole opening 26 shaped to keep the alignment of the clip as it enters the jaws.

The pitch of the spring 14 should equal the horizontal distance between the clip actuation bosses when the clip is oriented such that it has the smallest profile when seen from a distal viewing orientation. This will allow it to be advanced by the coil without binding.

Each clip assists in pushing the distally adjacent clip along its path. The last clip is pushed along by a disk 20 which remains within the assembly.

The device could be configured as a reloadable, reusable device into which the user preloads several clips, or alternatively as a single, pre-loaded disposable device that would lock onto a reusable handle. The apparatus as described could having a longer shaft to hold more clips, and could possibly also be slightly flexible through the use of compliant materials in the outer sleeve and orientation prongs.

What is claimed is:

1. A clip applier comprising:
an elongate shaft having a lumen proportioned to receive a plurality of surgical ligating clips;
a jaw assembly on a distal end of the shaft; and
a screw member rotatable within the lumen to sequentially advance surgical ligating clips disposed within the lumen towards the jaw assembly.

2. The clip applier of claim 1, wherein the screw member comprises a helical coil.

3. The clip applier of claim 1, further include a rail member positioned within the coil such that a clip advanced into the jaw assembly passes through a channel of the rail member.

4. The clip applier of claim 1, further including a track in the jaws, the track positioned to receive a clip passing from shaft into the jaw assembly.

5. A method of applying surgical clips to tissue, comprising:
positioning a plurality of surgical ligating clips within a lumen of a shaft;
rotating a screw member within the lumen to advance a clip towards a jaw assembly at a distal end of the shaft;
advancing the clip into the jaw assembly, the jaw assembly including a pair of jaw members;
with the jaw members in an opened position, positioning the jaw members adjacent to tissue to be clipped;
closing the jaw members to close the clip on the tissue; and
rotating the screw member to advance a second clip towards the jaw assembly.

6. The method of claim 5, wherein the screw member comprises a helical coil.

7. The method of claim 5, wherein advancing the clip towards the jaw assembly comprises advancing the clip through a channel of a rail member positioned within the coil.

8. The method of claim 5, wherein the jaw assembly includes a track, and wherein advancing the clip into the jaw assembly includes causing the track to receive a clip passing from the shaft into the jaws.
